(19) United States
(12) Patent Application Publication
(10) Pub. No.: US 2007/0100200 A1
(43) Pub. Date: May 3, 2007

(54) Capsule type medical device

(75) Inventors: Akihiro Suzuki, Sagamihara-shi (JP); Kazuo Tansho, Tokyo (JP); Yoshito Katagiri, Tokyo (JP); Tohru Tominami, Tokyo (JP)

Correspondence Address: COHEN, PONTANI, LIEBERMAN & PAVANE, 551 FIFTH AVENUE, SUITE 1210, NEW YORK, NY, 10176, US

(73) Assignee: Konica Minolta Photo Imaging, Inc., Tokyo (JP)

(21) Appl. No.: 11/585,425

(22) Filed: Oct. 23, 2006

(30) Foreign Application Priority Data
Oct. 27, 2005 (JP) JP2005-312493
Nov. 16, 2005 (JP) JP2005-331304
Dec. 19, 2005 (JP) JP2005-364557

(51) Int. Cl. A61B 1/00

(52) U.S. Cl. 600/101

(57) ABSTRACT

A capsule type medical device to conduct an operation in an internal organic portion of a body, is provided with a capsule body to move in the internal organic portion; and an operating member incorporated in the capsule body and to conduct the operation in the internal organic portion. The capsule body includes a bellow section formed on a peripheral surface of the capsule body such that the bellow section stretches or contracts in a longitudinal direction of the capsule body.

This application is based on Japanese Patent Application No. 2005-312493 filed on Oct. 27, 2005, No. 2005-331304 filed on Nov. 16, 2005, and No. 2005-364557 filed on Dec. 19, 2005, in Japanese Patent Office, the entire content of which is hereby incorporated by reference.

### BACKGROUND OF THE INVENTION

The present invention relates to a capsule type medical device for image picking-up in the organs, when mainly, a person takes down, and it moves in a predetermined organ.

Conventionally, in order to inspect the internal organs, a tubular endoscopic apparatus which has an image pick-up lens and image pick-up element at the leading edge and is inserted from the mouth or anus, is well known. Because in this endoscopic apparatus, while the inspection of the internal organs is carried out, the tubular member is continued to be inserted from the outside to the internal organs, the discomfort feeling or pain is not small.

In order to decrease the such a load of tested subject, recently, the capsule type medical device which the tested subject can take down a single item is developed. The capsule type medical device houses a micro image pick-up apparatus and is structured such that the picked-up image is transmitted to external devices by the radio, and after a predetermined organ is image picked-up, it is discharged from the anus naturally.

Relating to this capsule type medical device, many patent journals are disclosed.

For example, the first capsule whose outer shape is small and the second capsule whose outer shape is large are connected by a tube, and when takes down from the first capsule which is small, there is a capsule type medical device which can be taken down without any pain (refer to Patent Document 1).

Further, there is small sized photographic apparatus in which the capsule type medical device is structured by the capsule unit housing a camera part, the capsule unit housing an LED for illumination, and the capsule unit housing a communication part, and after the tested subject takes down 3 kinds of capsule units one by one, when they are automatically connected and integrated, and the tested subject can easily takes down them (refer to Patent Document 2).

Further, there is the capsule type medical device in which a camera providing respectively, for example, a telescopic lens and a wide lens at both ends, is independently provided, and its photographic direction is changed by being rotated in the body, (refer to Patent Document 3).

Further, there is the capsule type medical device in which a spiral protrusion is provided on its outer peripheral surface, and when it is rotated in the rotating magnetic field, the propelling speed is enlarged, and it can be smoothly propelled along the tubular path (refer to Patent Document 4).

Further, there is the capsule type medical device in which, when the spiral protrusion is provided on its outer peripheral surface and it is rotated in the rotating magnetic field, it can be introduced to the object part (refer to Patent Document 5).

Other than that, there is the capsule type medical device in which the medicine housed in the inside can be injected in the body (refer to Patent Document 6).

(Patent Document 1) Tokkai No. 2003-136387

(Patent Document 2) Tokkai No. 2004-329292

(Patent Document 3) Tokkai No. 2004-344655

(Patent Document 4) Tokkai No. 2005-52502

(Patent Document 5) Tokkai No. 2003-275170

(Patent Document 6) Tokkai No. 2005-185567

The capsule type medical device written in Patent Documents 1, 2 at least provides with the image pick-up lens for image picking-up organs, the image pick-up element which image forms the picked-up image and photoelectric converts it, LED which illuminates the organs to be picked-up at the time of image pick-up, image processing part for conducting a predetermined image processing on the photoelectric converted image, transmission part for transmitting outside the image of the image processing part, and receiving part for receiving the signal such as the image pick-up start from the outside, and sends the receives relating to the position or posture in the body.

Further, the capsule type medical device announced by the manufacturers is formed in the size of, for example, about diameter 10 mm, length 25 mm.

The capsule type medical device is naturally conveyed by the fibrillation in the organs in the human body shown in FIG. 1, however, the small bowel is narrow, and has many curved paths, and there is no security that this size capsule can surely pass the curved paths such as the small bowel. For example, in a part like a hair pin curve such as A part of the small bowel, depending on the largeness or length of the capsule type medical device, as shown in A part enlarged view of FIG. 2, the pass of the capsaule type medical apparatus becomes difficult.

For a time, when the capsule type medical device is caught in the curved par t on the way, it is necessary that the tubular member like as the conventional endoscope is inserted into the organs and the catching of the capsule type medical device is corrected, or takes out is, and to the tested subject, new discomfort feeling or pain is added.

### SUMMARY OF THE INVENTION

In view of such a problem, the present invention is attained, and the first object is to provide a capsule type medical device which is structured bendably in the length direction and the risk which is caught when it passes the narrow bending part such as the small bowel is decreased.

Further, when the capsule type medical device image picks up in the organs whose inner diameter is small such as the small bowel, the distance between the capsule type medical device and the inner wall of the organs, that is, the subject distance is not so much changed. However, when the capsule type medical device image picks-up in the organs whose inner diameter is large such as the stomach or the large bowel, the subject distance is changed by the position of the capsule type medical device. Accordingly, it is desirable that focus position can be adjusted corresponding to subject distance.

Further, depending on the object of the image pick-up, because there is a case of partially enlarged and image picked-up or case of image pick-up of the large range, it is desirable that the angle of view to be image picked-up can be changed. Relating to this point, in the capsule type medical device written In Patent Document 3, by the camera respectively provided at both ends, for example, the image pick-up of telephoto and the image pick-up of the wide can be conducted. However, when these two cameras are provided, the capsule type medical device becomes large, and the tested subject hardly takes down, and it becomes difficult that it passes the organs which is narrow and has many bending parts like as the small bowel. Further, it cost much.

In view of such a problem, the present invention is attained, and the second object of the present invention is to propose the capsule type medical device by which the distance between lenses is changed when it is a simple structure and a part of the image pick-up lens is moved in the optical axis direction, and the subject distance to be focused or the focal length can be changed.

Further, when the image of the organs of the tested subject picked-up by the capsule type medical device is observed by the inspection doctor by an external device, there is a case where an anxious disease is discovered at the periphery of the picked-up image. In this case, as described above, the lens may be changed to the wide lens and image picked-up so that the wide range is picked-up, however, there is a problem that the image becomes small and it becomes difficult to be observed.

Further, in the capsule type medical device written in Patent Document 4, by the external operation, it can be moved in the organs, however, it is difficult that the image pick-up direction is easily changed.

In view of such a problem, the present invention is attained, and the third object of the present invention is to propose the capsule type medical device by which the optical axis of the image pick-up lens can be easily tilted, and a part positioned in the slant direction can also be image picked-up.

Further, the capsule type medical device written in Patent Document 5 can move, because is moved by rotating, along the inner wall surface in the it inside of small bowel whose inner diameter is small, however, in the stomach whose inner diameter is large, when it is rotated along the inner wall surface, because there is a possibility that it is rotated in unexpected direction, it is difficult to introduce the objective position.

In view of such a problem, the present invention is attained, and the fourth object of the present invention is to propose the capsule type medical device in which, when it is structured so that it can move straightly, even when in the organ whose inner diameter is large, it can surely self-run to the objective position.

Other than that, the fifth object of the present invention is to propose the capsule type medical device by which can inject the medicine accommodated inside for cure in the body, or sample the body fluid for inspection.

Item 1. In the capsule type medical device having the image pick-up optical system for image picking-up in the organ in the body, the capsule type medical device characterized in that the bellow part stretching and contracting in the length direction is formed on the outer peripheral surface.

Item 2. The capsule type medical device written in Item 1, wherein the bellow part is formed in the stretched condition, and it is sheathed by a member soluble in the organs in the contracted condition.

Item 3. The capsule type medical device written in Item 1 or 2, wherein it has the bellow part which stretches and contracts in the length direction on the outer peripheral surface, and the first bellow member which at least sheathes the image pick-up lens, and the second bellow member which has the bellow part stretching and contracting in the length direction on the outer peripheral surface, and at least sheathes the image processing section for image processing the image picked-up by the image pick-up lens.

Item 4. The capsule type medical device written in Item 3, wherein, the member at least holding the image pick-up lens, and the member at least holding the image processing section, are connected by the universal joint.

Item 5. In the capsule type medical device having the image pick-up optical system for image picking-up in the body, the capsule type medical device which characterized in that it has the image pick-up optical system structured by the first lens and the second lens, and the sheath member at whose leading edge part, the first lens is formed, and which sheathes the second lens, and on whose outer peripheral surface, the bellow part stretching and contacting in the length direction is formed, and a drive member for stretching and contacting the bellow part, and when the drive member is driven and the bellow part is stretched and contacted, and the first lens is moved in the optical axis direction, the distance between lenses of the first lens and the second lens is changed.

Item 6. The capsule type medical device written in Item 5, wherein, when the distance between lenses of the first lens and the second lens is changed, the subject distance to be focused is changed.

Item 7. The capsule type medical device written in Item 5, wherein, when the distance between lenses of the first lens and the second lens is changed, the focal distance of the image pick-up optical system is changed.

Item 8. In the capsule type medical device having the image pick-up optical system for image picking-up in the body, the capsule type medical device which is characterized in that: it has a front part housing the image pick-up optical system and image pick-up element, the rear part housing the image processing section for processing the picked-up image, and the joint rotatably connecting the front part and the rear part, and the bellow part which at least sheathes the joint, connected to the front part and rear part and stretches and contracts in the length direction, the drive part is driven, and when a predetermined part in the peripheral direction of the bellow part is contracted and the other part to the predetermined part is stretched, the axis center of the front part to the axis center in the length direction of the rear part is tilted in the predetermined direction.

Item 9. The capsule type medical device written in Item 8, wherein the joint is a universal joint.

Item 10. The capsule type medical device written in any one Item of Items 5-9, wherein the micro-mini motor for driving the drive member is provided.

Item 11. The capsule type medical device characterized in that: it has the bellow part stretching and contracting in the length direction on the outer peripheral surface, the bellow part is composed of a plurality of bellow groups, and when a predetermined bellow group is individually stretches and contracts, it self-run.

Item 12. The capsule type medical device written in Item 11, wherein the movement in which only the first bellow group positioned at the leading edge of the advancing direction stretches from the contract condition of all bellow groups and the leading edge part is moved forward, next, the first bellow group is contracted, and the second bellow group positioned at back of the first bellow group is stretched, in succession, when the second bellow group is contracted, the third bellow group positioned at back of the second bellow group is stretched, is successively repeated, and after the n-bellow positioned at the trailing edge of the advancing direction is stretched, when it is contracted, the trailing edge part advances forward.

Item 13. The capsule type medical device written in Item 11 or 12, wherein the total length of the bellow groups in the stretched condition is shorter than the total length of the bellow groups in the contracted condition at the time of standstill.

Item 14. The capsule type medical device written in any one Item of Items 11-14, wherein the member included by the bellow part is divided into at least two units, and each of units is connected by the universal joint, and bends around the universal joint.

Item 15. The capsule type medical device written in Item 14, wherein it has the drive member for stretching and contracting the bellow part, and when drives the drive member, contracts a predetermined part in the peripheral direction of the bellow part, and stretches the other part to the predetermined part, the axis center of the other unit to the axis center in the length direction of one unit is bent around the universal joint.

Item 16. The capsule type medical device written in any one Item of Items 11-15, wherein it is provided with at least the image pick-up lens, illumination part for illuminating the subject, image pick-up element for photo-electrically converting the picked-up image, image processing section for image processing the photo-electrically converted image, and transmission and reception section for transmitting the image processed image to the external devices.

Item 17. The capsule type medical device written in any one Item of Items 11-16, wherein it is provided with the conductive high polymer actuator for driving the bellow group.

Item 18. The capsule type medical device written in any one Item of Items 11-16, wherein it is provided with the shape memory alloy for driving the bellow group.

Item 19. The capsule type medical device which is characterized in that: it is provided with the bellow part which is provided on the outer peripheral surface and stretches and contracts in the length direction, drive part for driving the bellow part, accommodation part connected to the bellow part, accommodates inside the medicine, and having the communicating hole communicating to the outside, and when the drive part is driven in the body, and the bellow part in the stretched condition is contracted, the medicine accommodated in the accommodation part is injected from the communicating hole.

Item 20. The capsule type medical device which is characterized in that: it is provided with the bellow part which is provided on the outer peripheral surface and stretches and contracts in the length direction, drive part for driving the bellow part, accommodation part connected to the bellow part, accommodates inside the sampled body fluid, and having the communicating hole communicating to the outside, and when the drive part is driven in the body, and the bellow part in the contracted condition is stretched, the body fluid is sampled into the accommodation part from the communicating hole.

Item 21. The capsule type medical device written in Item 19 or 20, wherein it is provided with at least the image pick-up lens, illumination part for illuminating the subject, image pick-up element for photo-electrically converting the picked-up image, image processing section for image processing the photo-electrically converted image, and transmission section for transmitting the image processed image to the external devices.

Item 22. The capsule type medical device written in any one Item of Items 19-21, wherein the drive part has the micro-mini motor.

According to the capsule type medical device written in Item 1, because the bellow part stretching and contracting in the length direction is formed on the outer peripheral surface, it is bendable in the length direction, even when the capsule type medical device is moved in the body by the fibrillation, and passes the place which is narrow like as the small bowel and bent many times, different from the conventional capsule type medical device, the risk to be caught is small.

According to the capsule type medical device written in Item 5, because the distance between lenses of the first lens and the second lens can be changed by the operation from the outside the body, the subject distance to be focused can be changed to near distance or far distance, or composed focal distance of the first lens and the second lens can be changed to the short focal distance or long focal distance.

According to the capsule type medical device written in Item 8, by the operation from the outside the body, while the capsule type medical device is fixed, the optical axis of the image pick-up optical system can be tilted in the desired direction.

According to the capsule type medical device written in Item 11, it can self-run by a simple structure, and as the conventional technology, because the movement is not rotation movement, but the straight movement, even when the capacity is large place, it can surely self-run and approach to the objective position.

According to the capsule type medical device written in Item 19, the medicine accommodated inside can be directly injected to the disease for cure.

According to the capsule type medical device written in Item 20, the body fluid in a predetermined organ can be sampled for inspection.

### BRIEF DESCRIPTION OF THE DRAWINGS

FIG. 1 is a view in the organs of the human body.

FIG. 2 is an enlarged view in which the capsule type medical device is caught at A part of FIG. 1.

FIG. 3 is a sectional view of the capsule type medical device before use.

FIG. 4 is a sectional view of the capsule type medical device after it is taken down.

FIG. 5 is a sectional view of the bent capsule type medical device.

FIG. 6 is an enlarged view in which the capsule type medical device passes A part of FIG. 1.

FIG. 7 is a sectional view of the capsule type medical device when the subject of the near distance is image picked-up from the front.

FIG. 8 is a sectional view of the capsule type medical device when the subject of the far distance is image picked-up from the front.

FIG. 9 is a sectional view of the capsule type medical device when the image pick-up optical system is tilted for image picking-up.

FIG. 10 is a sectional view of the capsule type medical device in the stationary condition.

FIG. 11 is a view showing the sequence in which the capsule type medical device self-runs.

FIG. 12 is an enlarged view when as the actuator, the conductive high polymer actuator is used.

FIG. 13 is an operation principle view of the conductive high polymer actuator.

FIG. 14 is an enlarged view when as the actuator, the shape memory alloy is used.

FIG. 15 is a side view and an upper surface view of the capsule type medical device which houses one universal joint and self-runs.

FIG. 16 is an upper surface view of the capsule type medical device which houses two universal joints and self-runs.

FIG. 17 is a sectional view of the capsule type medical device for injecting the medicine.

FIG. 18 is a sectional view of the capsule type medical device for sampling the body fluid.

### DETAILED DESCRIPTION OF THE PREFERRED EMBODIMENT

### The First Embodiment

Referring to the drawing, the first embodiment according to the capsule type medical device of the present invention will be described.

Initially, based on FIG. 3, the structure of the capsule type medical device will be described, however, FIG. 3 is a sectional view of the capsule type medical device before use.

The present capsule type medical device 101 is structured by the front part 102, rear part 103, and the connection part 104 connecting the front part 102 and the rear part 103.

In the front part 102, the image pick-up lens 121 structured in the wide angle, and for image picking-up in the organs of the person, image pick-up element 122 composed of CCD or CMOS, and for image-forming the image, picked-up by the image pick-up lens 121 and for photoelectric converting, and a plurality of illumination members 123 composed of LED, and for illuminating the organ to be image picked-up at the time of image picking-up, are housed, and they are held by the connection member 141.

Then, the trailing edge part 125c of the front body 125 (the first bellow member) having the flexible bellow part 125a on the outer peripheral surface is fixed to the connection member 141 of the connection part 104, and the front body 125 sheathes the inside image pick-up element 122 in the water-tight condition. Hereupon, the leading edge part 125b of the front body 125 is formed into sphere-like so that it easily passes in the organ, and at least the leading edge part 125b is formed into transparent and equal thickness, and structured so that it does not hinder the image picking-up.

Hereupon, the leading edge part 125b is formed so that the wall thickness is changed from the central part to the peripheral part, and is made a convex lens or concave lens, and may also structure the image pick-up optical system together with the image pick-up lens 121.

In the rear part 103, the image processing section 131 for processing the image photoelectric converted by the image pick-up element 122, transmission and reception section 132 for transmitting the image processed in the image processing section 131 to outside the body by the wireless, and for receiving the wireless signal from outside the body, and the battery 133 for supplying the electric source to each circuit part are housed, and they are held by the rear part holding member 142.

Then, the leading edge part 135c of the rear body 135 (the second bellow member) having the flexible bellow part 135a on the outer peripheral surface is fixed to the connection member 141 of the connection part 104, and the rear body 135 sheathes the inside image processing section 131 in the water-tight condition. Hereupon, the trailing edge part 135b of the rear body 135 is also formed into sphere-like so that it easily passes in the organ.

In the connection part 104, the spherical member 143 having the spherical part 143a formed into spherical, is integrally connected to the connection member 141, spherical part 143a is inserted into spherical hole 142a provided in the rear part holding member 142, and the top part of the spherical part 143a is held by the holding plate 144 fixed to the rear part holding member 142.

By the above description, because the spherical part 143a is rotatable to the spherical hole 142a, the universal joint is structured, and the connection member 141 is freely rotated in all directions to the rear part holding member 142.

Hereupon, as the universal joint, it is not limited to such a structure, but, for example, the first axis is rotatably passed into the C-shaped first member, the second axis is rotatably passed into the other C-shaped second member, and the second axis is rotatably passed into the hole provided in the peripheral wall center of the first axis, and the first axis and the second axis are crossed at right angle, and it may also be the universal joint in which the second member is 3-dimensionally rotated to the first member.

The capsule type medical device 101 is structured as described above, further, before use, in the condition that the bellow part 125a of the front body 125 and the bellow part 135a of the rear body 135 are contracted, they are sheathed by the sheath member 151, so that the capsule type medical device 101 is easily taken down. Hereupon, the sheath member 151 is formed of the material which is dissolved by the body fluid in the organ, for example, the gelatin formed of the bone of caw and the skin of pig, or the oblate formed of the agar and starch. Further, because the front body 125, rear body 135 are molded in the condition that the bellow parts 125a, 135a are stretched, when the sheath member 151 which sheathes is dissolved, the bellow parts 125a, 135a are released from the contracted condition, and automatically stretched.

Next, the mode of operation of the capsule type medical device 101 will be described.

Initially, the tested subject takes down the capsule type medical device 101 from the direction of the front part 102. In this time, because, in the capsule type medical device 101, the bellow parts 125a, 135a of the front body 125, rear body 135 are in the contracted condition, and the total length is short, and the bellow parts 125a, 135a having the concave and convex are sheathed, the resistance for taking down is small.

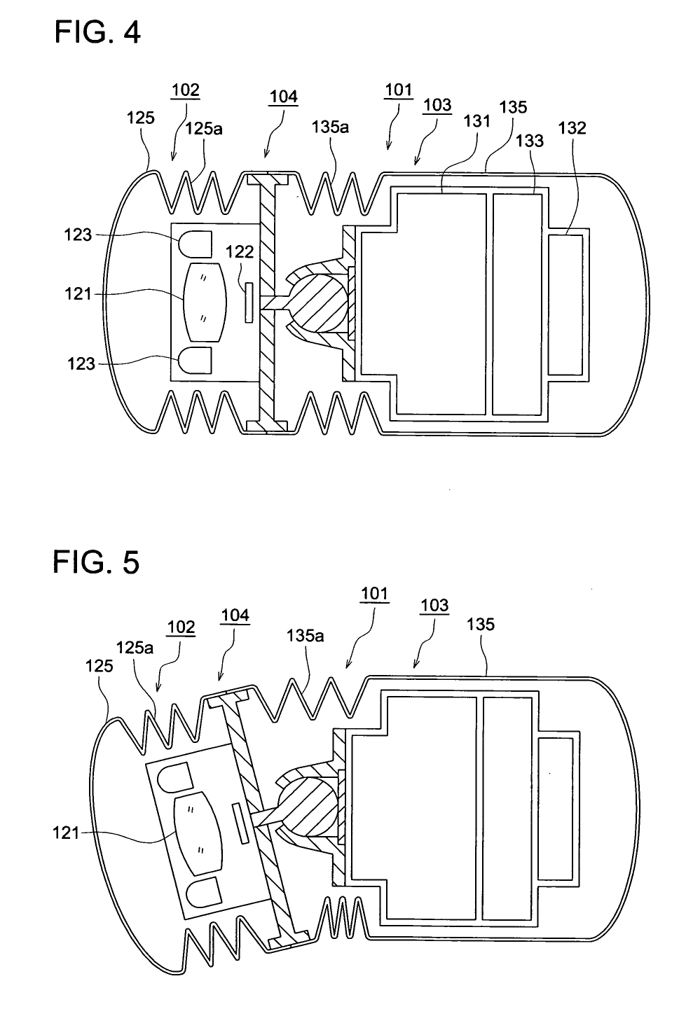

The taken down capsule type medical device 101 passes the oesophagus, when it arrives at the stomach, the sheath member 151 is dissolved by the gastric juice. Hereby, the contracted bellow parts 125a, 135a of the front body 125, rear body 135 are as shown in FIG. 4, released from the contracted condition, automatically stretched, and becomes the condition at the time of molding. Accordingly, the capsule type medical device 101 becomes narrow and long, and easily directed in the advancing direction in the organ, and is easily moved by the fibrillation. The capsule type medical device 101 is moved to the stomach, small bowel, and large bowel by the fibrillation. Then, from the transmission and reception section 132 of the capsule type medical device 101, the signal except the image signal is continuously transmitted to outside the body. Accordingly, by the external devices, not shown, composed of the receiving part, data processing part, display part and input part, the inspection doctor can grasp the position in the body of the capsule type medical device 101.

In the case where the capsule type medical device 101 arrives at a predetermined organ, when the inspection doctor operates the external device for image picking-up this organ and transmits the image pick-up signal, the transmission and reception section 132 of the capsule type medical device 101 receives the signal. Then, the signal received by the transmission and reception section 132 is transmitted to the image processing section 131, and by the control of the control section provided in the image processing section 131, the image pick-up of the organ is conducted. Hereupon, when image pick-up, the illumination member 123 is turned on, and illuminates the organ.

The image of the organ which is the subject, is image formed on the image pick-up element 122 by the image pick-up lens 121, and photoelectric converted. Then, the photoelectric converted signal is A/D conversion processed in the image processing section 131 and becomes the image signal, and compression processed and temporarily stored in the memory. The image signal in the memory is modulated in the transmission and reception section 132, and transmitted to the external device outside the body in the electric wave through the antenna.

Accordingly, the inspection doctor can observe the image of the organ of the tested subject by the external device or store in a predetermined memory mean.

Hereupon, in the structure described above, when the capsule type medical device 101 is not operated, so that the battery 133 does not consume wastefully, the power source switch interlocking with the stretch and contraction of the bellow part 135a of the rear body 135, is provided, only when the sheath member 151 is dissolved in the body and the bellow part 135a is stretched, the power source switch may also be turned on.

Further, when from the coil arranged outside the body, the electric power is supplied to the power receiving coil provided in the capsule type medical device 101 by the electromagnetic induction, the battery 133 is not necessary.

Further, the posture sensor is housed in the rear part 103, and the posture of the capsule type medical device 101 may also be transmitted to the external device by the transmission and reception section 132.

In this manner, the capsule type medical device 101 is moved in the body by the fibrillation, however, when it passes a place which is narrow and bent many times like as the small bowel, there is a possibility that the conventional capsule type medical device is caught.

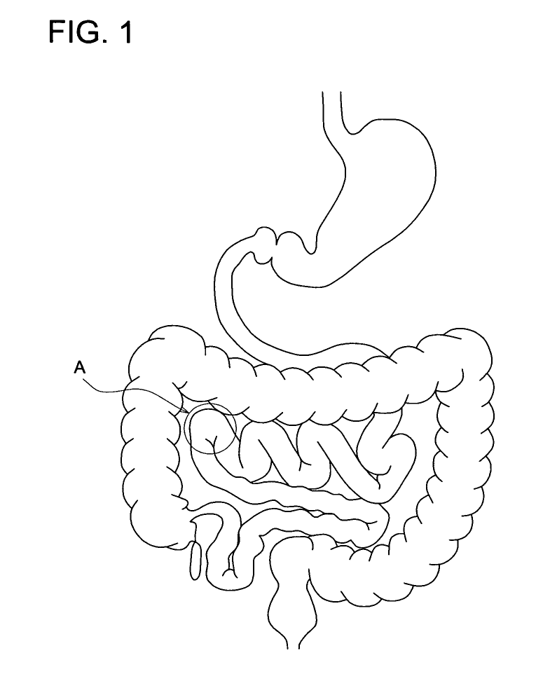

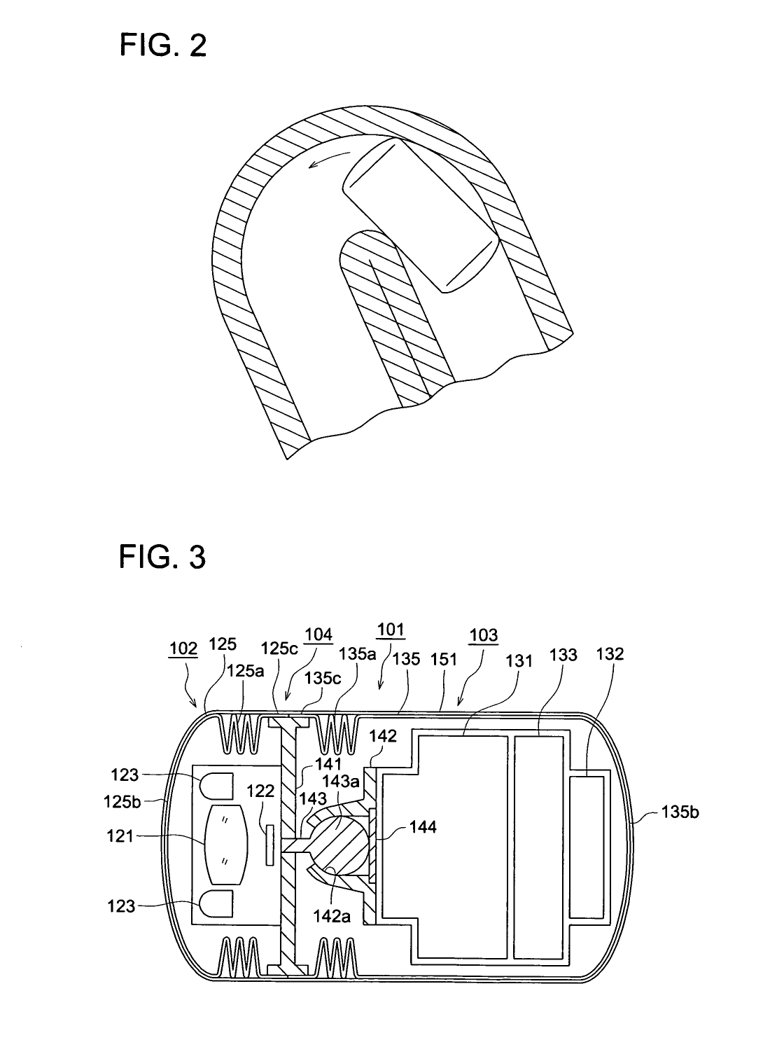

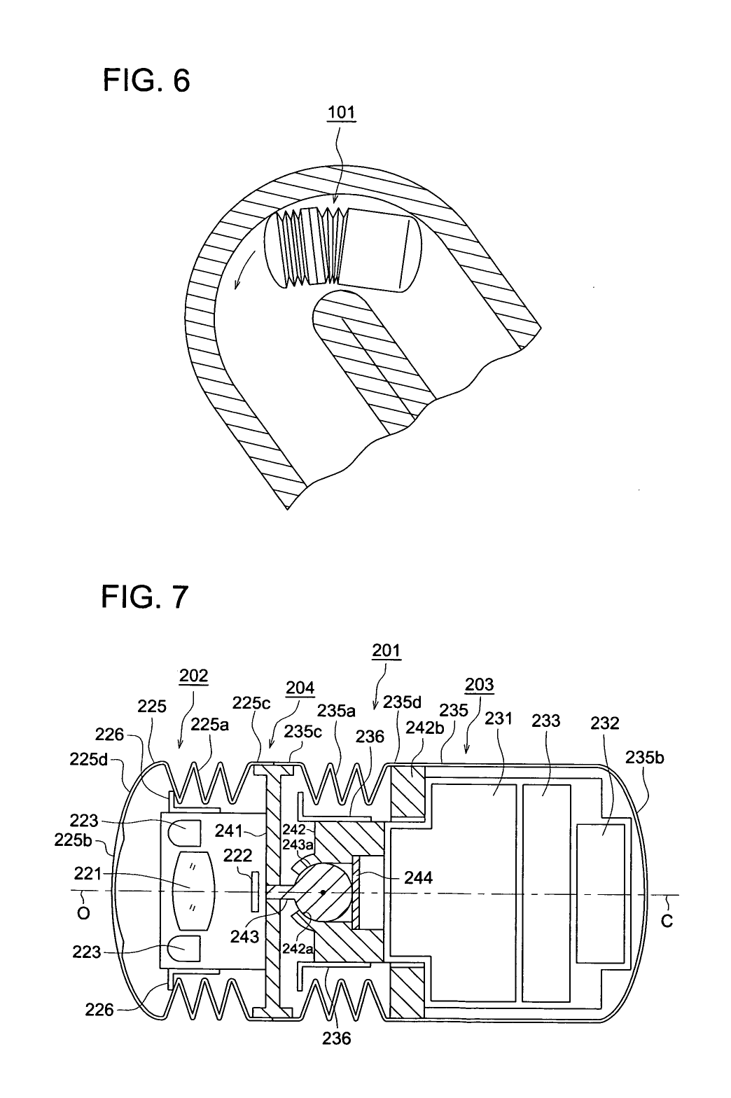

However, in the present capsule type medical device 101, a cover sheathing the inside member of the front part 102 and the rear part 103 is structured by the front body 125 having the bellow part 125a and the rear body 135 having the bellow part 135a, and the inside member is accommodated by being divided into the front part 102 side and the rear part 103 side, and because they are connected by the universal joint of the connection part 104, as shown in FIG. 5, the front part 102 can freely rotate in all directions to the rear part 103. Accordingly, when it is conveyed to the bending part of the organ like A shown in FIG. 1, as shown in the enlarged view of FIG. 6, because the capsule type medical device 101 itself is bent corresponding to the curvature of the bending part, it can smoothly pass without being caught. Hereupon, for the bellow part of the capsule type medical device 101, the condition shown in FIG. 4 is the normal condition, however, when it passes the bending part, for example as shown in FIGS. 5, 6, because one hand bellow part is stretched, the other bellow part is compressed, when the capsule type medical device 101 passes the bending part and arrives at a straight line part, it naturally restores to a condition of FIG. 4.

Further, although the below part 125a contributes to an action to prolong the total length in the organ, because it does not contribute to the passage of the bending part, it can also be neglected.

Further, two or more universal joints may also be provided, and in this case, 3 sets or more bellow parts are provided.

Further, when the gap between the front body 125, rear body 135 for sheathing the inside member and the inside member, is enough, even when the connection section 104 does not necessarily have the universal joint, it bends in all directions by the bellow parts 125a, 135a. Further, in this case, the bellow part may also be one set or 3 sets or more, further, the bellow parts may also be formed on all of the outer peripheral wall.

### The Second Embodiment

Referring to the drawings, the second embodiment according the capsule type medical device of the present invention will be described below.

Initially, based on FIG. 7, the structure of the capsule type medical device will be described, and FIG. 7 is a sectional view of the capsule type medical device when the subject of the near distance is image picked-up in the front.

The present capsule type medical device 201 is structured by the front part 202, rear part 203, and the connection part 204 connecting the front part 202 and the rear part 203.

In the front part 202, the second lens 221, the image pick-up element 222 for image forming and photoelectric converting the picked-up image by the image pick-up optical system which will be described later, and a plurality of illumination members 223, composed of LED, for illuminating the organ to be picked-up at the time of the image pick-up, are housed, and they are held by the connection member 241 of the connection part 204.

Then, the trailing edge part 225c of the front body 225 having the flexible bellow part 225a on the outer peripheral surface is fixed to the connection member 241, and the sheath member 25 sheathes the housed second lens 221 in the water-tight condition. Further, on the leading edge part of the front body 225, the first lens 225b integrally molded with the front body 225 is formed, and the image pick-up optical system is structured by the first lens 225b and the second lens 221.

Further, the leading edge part 225d of the front body 225 is formed into spherical, so that it can easily pass in the organ, and at least a part in which the first lens is formed, is formed into transparent.

Hereupon, the first lens 225b may also not necessarily be integrally molded with the front body 225, but, may also be independently formed and fixed to the front body 225. In this case, it is more desirable that it is fixed to a position at which the relative interval to the second lens 221 can be changed.

Further, in the front part 202, a plurality of drive members 226 for stretching and contracting the bellow part 225a of the bellow member 225 from inside, are provided on the periphery, and when the drive member 226 goes forward and backward in the direction of optical axis O, the bellow part 225a is stretched and contracted in the direction of the optical axis O. In this case, a plurality of drive members 226 simultaneously integrally goes forward and backward. Further, as the actuator for moving the drive member 226, it is desirable that the micro-mini motor is used.

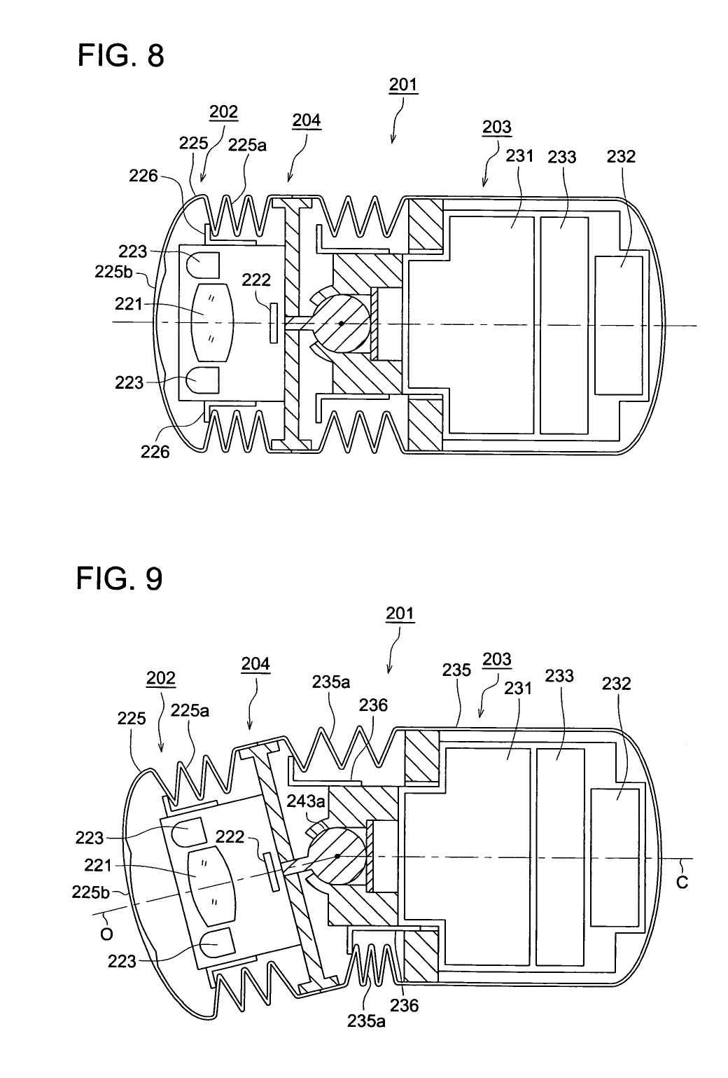

Then, when the drive member 226 is moved to the direction of the image pick-up element 222, the bellow part 225a is compressed, and becomes the condition of FIG. 8. In this case, the bellow part 225a is molded in the condition that it is a little stretched condition from the condition of FIG. 7, and when the drive member 226 presses it, the bellow part 225a is shrunk.

Hereupon, it may also be structured that the bellow part 225a is molded in the condition that it is shrunk, inverse to 2 in FIG. 7, the drive member 226 presses it from the side of the image pick-up element 222, and the bellow part 225a is stretched.

In the rear part 203, the image processing section 231 for processing the image photoelectric converted by the image pick-up element 222, the transmission and reception section 232 having the antenna, for transmitting the image processed in the image processing section 231 in the wireless to the outside of body, and for receiving the wireless signal from the outside of body, and the battery 232 for supplying the power source to each of circuit parts are housed, and they are held by the rear part holding member 242.

Then, the leading edge part 235c of the rear body 235 having the flexible bellow part 235a on the outer peripheral surface is fixed to the connection member 241 of the connection part 204, and the intermediate part 235d of the rear body 235 is fixed to the trailing edge part 242b of the rear part holding member 242, and the rear body 235 sheathes the inside image processing section 231 in the water-tight condition. Hereupon, the trailing edge part 235b of the rear body 235 is also formed into spherical so that it easily passes in the organ.

Further, in the rear part 203, the drive member 236 for moving the bellow part 235a of the rear body 235 from the inside, are provided on the periphery at the plural places, and by the control of the control section provided in the image processing section 231, each of drive members 236 independently goes forward and backward. Hereby, desired drive member 236 is moved backward, presses the bellow part 235a and makes shrunk. On the one hand, the other drive member 236 is moved forward, because it retreats from the bellow part 235a, the bellow part 235a is stretched. Hereupon, in the normal condition, each drive member 236 retreats forward from the bellow part 235a, and the bellow part 235a is in the condition at the time of molding. Further, as the actuator for driving each of drive members 236, it is desirable that the micro-mini motor is used.

In the connection part 204, the spherical member 243 having the spherically formed spherical part 243a is integrally connected to the connection member 241, the spherical part 243a is inserted into a spherical hole 242a provided in the rear par t holding member 242, and the top part of the spherical part 243a is held by the holding plate 244 fixed to the rear part holding member 242.

From the above description, because the spherical part 243a is rotatable to the spherical hole 242a, the universal joint is structured, and the connection member 241 is freely rotated to the rear part holding member 242 in all directions.

Hereupon, as the universal joint, it is not limited to such a structure, but the universal joint in which, for example, the first axis is rotatably inserted into C-shaped first member, and the second axis is rotatably inserted into other C-shaped second member, and the second axis is rotatably inserted into the hole provided in the peripheral wall center of the first axis, and crossed at right-angle, and the second member rotates 3-dimensionally to the first member, may also be allowable.

Other than that, relating to the battery, when the electric power is supplied from the coil arranged outside the body to the power receiving coil provided in the capsule type medical device 201 by the electromagnetic induction, the battery is unnecessary.

Further, the posture sensor is housed in the rear part 203, and the posture of the capsule type medical device 201 may also be transmitted to the external device by the transmission and reception section 232.

Next, the mode of operation of the capsule type medical device 201 will be described.

Initially, the tested subject takes down the capsule type medical device 201 from the direction of the front part 102.

Then, The capsule type medical device 201 is moved to the stomach, small bowel, and large bowel by the fibrillation. From the transmission and reception section 232 of the capsule type medical device 201, the signal except the image signal is continuously transmitted to outside the body. Accordingly, by the external devices, not shown, composed of the receiving part, data processing part, display part and input part, the inspection doctor can grasp the position in the body of the capsule type medical device 201.

Hereupon, depending on the case, there is a case where the capsule type medical device 201 is inserted from the anus.

In the case where the capsule type medical device 201 arrives at a predetermined organ, when the inspection doctor operates the external device for image picking-up this organ and transmits the image pick-up signal, the transmission and reception section 232 of the capsule type medical device 201 receives the signal. Then, the signal received by the transmission and reception section 232 is transmitted to the image processing section 231, and by the control of the control section provided in the image processing section 231, the image pick-up of the organ is conducted. Hereupon, when image pick-up, the illumination member 223 is turned on, and illuminates the organ.

The image of the organ which is image picked-up, is image formed on the image pick-up element 222 by the image pick-up lens 221, and photoelectric converted. Then, the photoelectric converted signal is A/D conversion processed in the image processing section 231 and becomes the image signal, and compression processed and temporarily stored in the memory. The image signal in the memory is modulated in the transmission and reception section 232, and transmitted to the external device outside the body in the electric wave through the antenna.

Accordingly, the inspection doctor can observe the image of the organ of the tested subject by the external device or store in a predetermined memory mean.

Further, in the case where the organ is image picked-up, in the capsule type medical device 201 in the condition of FIG. 7, when the focus of the image is adjusted on the part of the near direction and the part of the far distance is not sharp, when the inspection doctor operates the external device and transmits the distance signal to the capsule type medical device 201, through the transmission and reception section 232, the control section in the image processing section 231 controls the micro-mini motor and moves the drive member 226. Then, a plurality of drive members 226 simultaneously press the bellow part 225a of the front body 225 and contract it, and changes from the condition of FIG. 7 to the condition of FIG. 8, and the first lens 225b is gone back on the side of the image pick-up element 222. Hereby, the distance between lenses of the first lens 225b and the second lens 221 is reduced, the far distance part of the image pick-up element 222 is focused, and the focus of the image becomes sharp.

In this manner, by the operation from the outside the body, the subject distance to be focused, can be changed.

Further, by the lens composition of the first lens 225b and the second lens 221, when the distance between lenses is changed, the focal distance can also be changed. Accordingly, for example, the condition of FIG. 7 is long focal distance, and the condition of FIG. 8 is short focal distance, and from the same position, it can be image picked-up at different angle of view.

Hereupon, when the drive member 226 is moved by the micro-mini motor, even when the photographing distance is changed, or the focal distance is changed, it can be changed in step-less or set predetermined step number.

### The Third Embodiment

Referring to the drawings, the third embodiment according the capsule type medical device of the present invention will be described below.

When the image of the organs of the tested subject is observed by the inspection doctor by an external device, there is a case where an anxious disease is discovered at the periphery of the picked-up image. In this case, as described above, the lens may be changed to the wide lens and image picked-up so that the wide range is picked-up, however, there is a problem that the image becomes small and it becomes difficult to be observed.

In this case, when the inspection doctor operates the external device and the tilt signal is transmitted to the capsule type medical device 201, the control section in the image processing section 231 controls the micro-mini motor by the transmission and reception section 232, and each drive member 236 is moved. Then, the drive member 236 positioned in the direction in which the image pick-up optical system is wanted to tilt is moved backward, and the bellow part 235a is pressed and contracted. Hereby, as shown in FIG. 9, the front part 202 rotates in the direction of the drive member 236 moved backward, around the spherical apart 243a. In this case, because to the contracted bellow part 235a, the other bellow part 235a placed at least 180° position is stretched, the drive member 236 in the vicinity of the stretched bellow part 235a is moved forward, and retreated from the bellow part 235a. Accordingly, while the capsule type medical device 201 is fixed, the axis center of the front part 202, that is, the optical axis O of the image pick-up optical system is tilted to the axis center C of the rear part 203, and the organ in the desired direction can be image picked-up.

Hereupon, the above structure is reversed, and the drive member 236 placed in the reversal position to the direction wanted to tilt the image pick-up optical system is moved forward, and the bellow part 235a may also be pressed and stretched. Hereby, the bellow part 235a positioned in the direction wanted to tilt the image pick-up optical system is contracted, and it is in the condition same as described above.

Further, when the drive member 236 is moved by the micro-mini motor, the image pick-up optical system can be tilted in the arbitrary direction at the arbitrary angle.

In this manner, only the image pick-up optical system is tilted in the desired direction without moving the capsule type medical device, and in that condition, further focused subject distance or focal distance can be changed.

Further, as described above, a fact that the front part 202 of the capsule type medical device 201 is tilted to the rear part 203, is not only effective for changing the photographing direction, but also effective when the capsule type medical device 201 passes the organ which is narrow and has many bending part.

Hereupon, in the above description, the bellow part 235a is formed in the rear body 235, however, the bellow part 235a is made the separated member from the rear body 235, and may also connect to the front part 202 and rear part 203.

Further, the capsule type medical device 201 described as above, has the bellow part 225a and the bellow part 235a, after the photographing direction is changed, the subject distance to be focused, or focal distance can be changed, however, only the bellow part 225a is provided, and it may also be allowable that the subject distance to be focused, or focal distance is only changed, or only the bellow part 235a is provided, and the photographing direction is only changed.

Further, as the actuator for driving the drive members 226, 236, it is not necessarily limited to the micro-mini motor, but the conductive high polymer actuator, shape memory ally, piezo-electric element may also be used.

Other than that, in the above description, the drive members 226, 236 of the capsule type medical device 201 positioned in the body of the tested subject, are operated by the communication from outside the body, however, the drive members 226, 236 may also be automatically driven. That is, CPU having the program for driving the drive members 226, 236 is housed in the capsule type medical device 201, and when a predetermined condition is satisfied, the drive members 226, 236 are automatically driven, and the subject distance or focal distance may also be changed.

### The Fourth Embodiment

Referring to the drawing, the fourth embodiment according to the capsule type medical device of the present invention will be described.

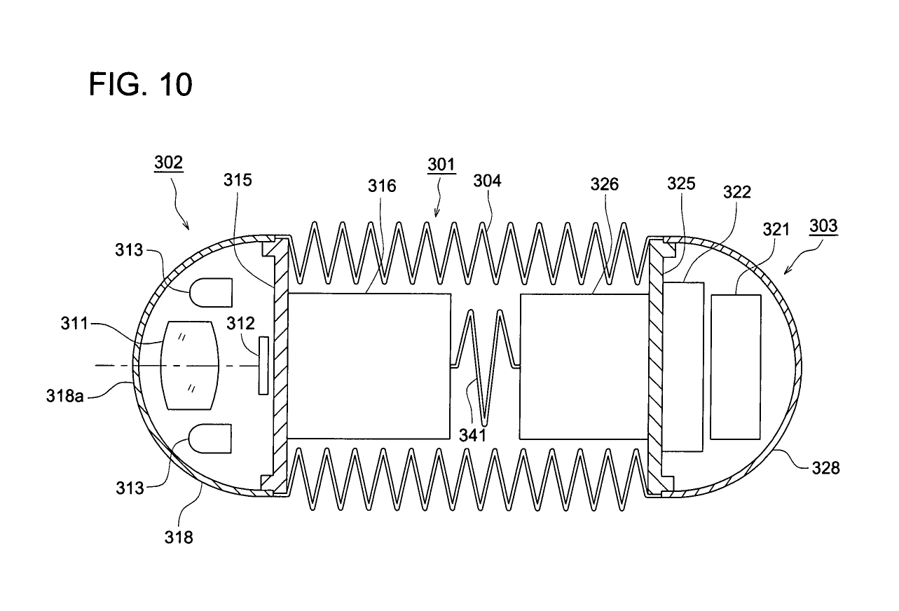

Initially, based on FIG. 10, the structure of the capsule type medical device will be described, however, FIG. 10 is a sectional view of the capsule type medical device in the stationary condition.

The present capsule type medical device 301 is structured by the front part 302, rear part 303, and the bellow part 304 connecting the front part 302 and the rear part 303.

In the front part 302, the image pick-up lens 311 for image picking-up in the organ of the person, image pick-up element 312 composed of CCD or CMOS, and for image-forming the image, picked-up by the image pick-up lens 311 and for photoelectric converting, and a plurality of illumination parts 313 composed of LED, and for illuminating the organ to be image picked-up at the time of image picking-up, are housed, and they are held in the font of the front part substrate 315. Further, in the rear of the front part substrate 315, the image processing section 316 for processing the image photoelectric converted by the image pick-up element 312 is arranged, and held by the front part substrate 315.

Then, the image pick-up lens 311 held by the front part substrate 315 is sheathed by the front body 318 in the water-tight condition. Hereupon, at least leading edge part 318a of the front body 318 is formed into transparent and equal in thickness, and structured so that it does not hinder the image pick-up.

In the rear part 303, the transmission and reception section 321 having the antenna, for transmitting the image processed in the image processing section 316 in the wireless to the outside of body, and for receiving the wireless signal from the outside of body, and the battery 322 for supplying the power source to each of circuit parts are housed, and they are held by the rear part substrate 325. Further, in the front part side, which is the reversal side of the rear part substrate 325, the control section 326 for controlling the timing of the image pick-up, or for controlling for self-running the capsule type medical device which will be described later, is arranged, and held by the rear part substrate 325.

Then, the transmission and reception section 321 held by the rear part substrate 325 is sheathed by the rear body 328 in the water-tight condition.

Further, the image processing section 316 of the front part, the control section 326 of the rear part 303, the battery 322 and the transmission and reception section 321 are flexibly and electrically connected by the connection member 341 composed of a flexible print substrate or a plurality of lead wires.

Herein, when the bellow part 304 is compressed as in FIG. 10, the maximum outer diameter of the bellow part 304 is formed larger than the outer diameter of the front body 318 and the rear body 328.

Further, although it is not shown in FIG. 10, the bellow part 304 is composed of a plurality of bellow groups as will be described later, and the actuator for respectively stretching and contracting a predetermined bellow group is provided in the inside of the bellow part 304.

Next, the self-running action of the capsule type medical device will be described.

Initially, when the tested subject takes down the capsule type medical device from the direction of the front part 302, the capsule type medical device passes the oesophagus, and arrives at the stomach, and is moved to the small bowel, and large bowel by the fibrillation. Then, from the transmission and reception section 321 of the capsule type medical device, the signal except the image signal is continuously transmitted to outside the body. Accordingly, by the external devices, not shown, composed of the receiving part, data processing part, display part and input part, the inspection doctor can grasp the position in the body of the capsule type medical device.

In the case where the capsule type medical device arrives at a predetermined organ, when the inspection doctor operates the external device for image picking-up this organ and transmits the image pick-up signal, the transmission and reception section 321 of the capsule type medical device receives this signal. Then, by the signal received by the transmission and reception section 321, the control section 326 controls and the image pick-up of the organ is conducted. Hereupon, when image pick-up, the illumination part 323 is turned on, and illuminates the organ.

The image of the organ which is the subject, is image formed on the image pick-up element 312 by the image pick-up lens 311, and photoelectric converted. Then, the photoelectric converted signal is A/D conversion processed in the image processing section 316 and becomes the image signal, and compression processed and temporarily stored in the memory. The image signal in the memory is modulated in the transmission and reception section 321, and transmitted to the external device outside the body in the electric wave through the antenna.

Accordingly, the inspection doctor can observe the image of the organ of the tested subject by the external device or store in a memory mean such as the hard disk or CD.

Herein, in the case where it is wanted that the capsule type medical device in the organ is self-run to the arbitrary position, and image pick-up is conducted, when the inspection doctor transmits the movement signal by the external device, the transmission and reception section 321 of the capsule type medical device receives this signal, and based on the signal, the control section 326 controls the actuator, not shown, and stretches and contracts the bellow group of the bellow part 304, and the capsule type medical device is self-run.

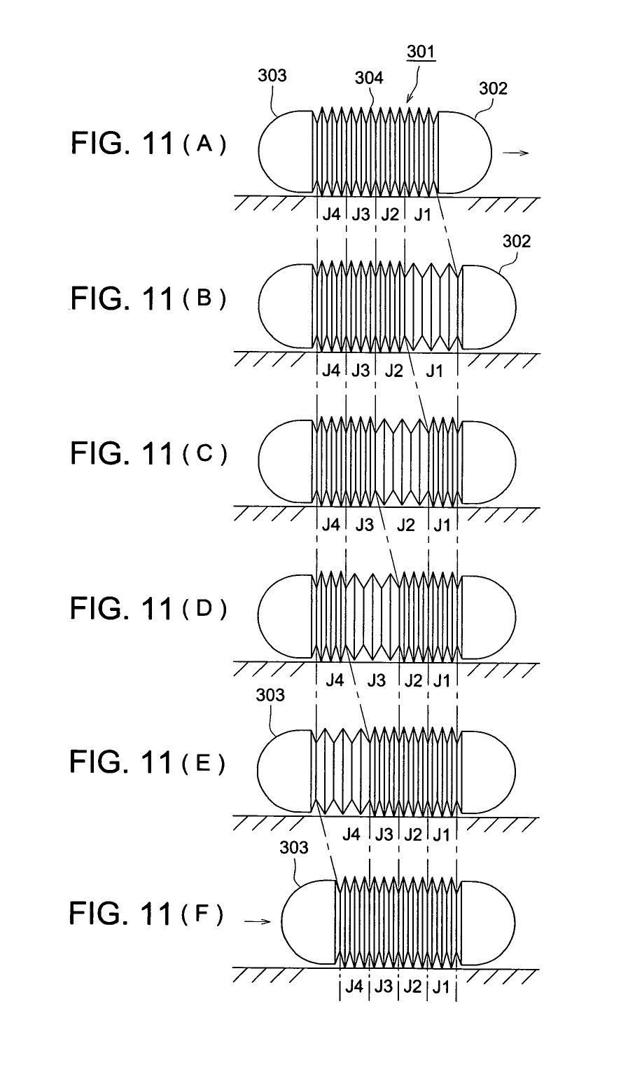

Based on FIG. 11, the sequence for self-running the capsule type medical device by this stretching and contracting of the bellow group, will be described.

FIG. 11 shows the condition that the capsule type medical device self-runs in the sequence of (A)-(F) to the right direction in the drawing.

The bellow part 304 of the capsule type medical device 301 shown in FIG. 11 is formed of 4 bellow groups J1-J4 each of which has 3 protrusions, and each of bellow groups independently stretches and contracts.

Initially, in FIG. 11(A), the capsule type medical device 301 is in the condition before self-running, and the bellow groups J1-J4 are all contracted and grounded.

Next, in FIG. 11(B), when only the leading edge bellow group J1 (the first bellow group) is stretched, the front part 302 (leading edge part) is moved forward. In this case, because the bellow groups J1-J4 are grounded, even when the bellow group J1 and the front part 302 are floated, they are held, and the capsule type medical device 301 does not fall forward.

In succession, in FIG. 11©, the stretched bellow group J1 is contracted, and the second bellow group J2 (the second bellow group) is stretched. In this case, even when the bellow group J2 is floated, because the bellow group J1 and bellow groups J3, J4 are grounded, the capsule type medical device 301 does not fall forward and backward.

In succession, in FIG. 11(D), the stretched bellow group J2 is contracted, and the third bellow group J3 (the third bellow group) is stretched. In this case, even when the bellow group J3 is floated, because the bellow groups J1, J2 and bellow group J4 are grounded, the capsule type medical device 301 does not fall forward and backward.

In succession, in FIG. 11(E), the stretched bellow group J3 is contracted, and the trailing edge bellow group J4 is stretched. In this case, even when the bellow group J4 is floated, because the bellow groups J1-J3 are grounded, the capsule type medical device 301 does not fall backward.

Finally, in FIG. 11(F), the stretched bellow group J4 is contracted, Hereby, the rear part 303 (trailing edge part) is moved forward, and the whole of the capsule type medical device 301 is moved forward from the condition of FIG. 11(A).

When such a moving motion is repeated, the capsule type medical device 301 surely advances by little and little.

Further, when the operation reversal to the above description is conducted, the capsule type medical device 301 is moved backward.

In this manner, by the external operation, the capsule type medical device 301 can be self-run to a predetermined position in the organ, and the image pick-up can be done at an adequate position.

Hereupon, the number of the bellow groups is not limited, and when the bellow group positioned at the most leading edge is the first bellow group, the n-th bellow group positioned at the most trailing edge is the n-th bellow group.

Further, the number of protrusions of the bellow group is not also limited.

Further, when the total length of the bellow group of stretched condition is shorter than the total length of the bellow group of the contracted condition at the time of stasis, it can stably and surely self-run.

Next, the actuator for moving the bellow group will be described.

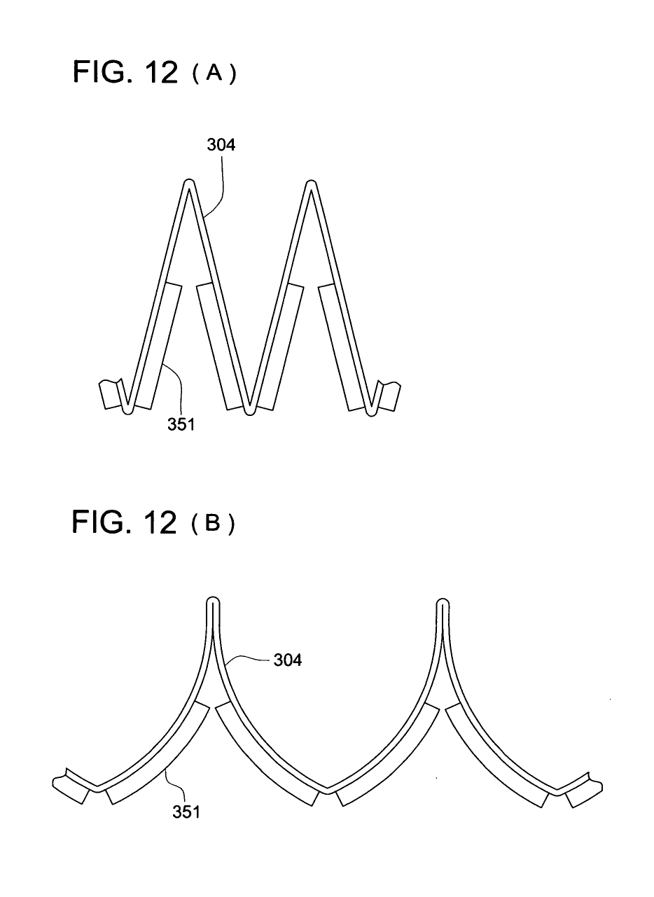

FIG. 12 is an enlarged view when the conductive high polymer actuator is used as the actuator, and FIG. 12(A) is a view of the condition that the bellow part is contracted, and FIG. 12(B) is a view of the condition that the bellow part is stretched.

In FIG. 12, the conductive high polymer actuator 351 is adhered to the bending each inside of the bellow part 304. When the voltage is not applied on this conductive high polymer actuator 351, the bellow part 304 is contracted as shown in FIG. 12(A), however, when the voltage is applied on the conductive high polymer actuator 351, because the conductive high polymer actuator 351 is bent, the bellow part 304 is stretched as shown in FIG. 12(B).

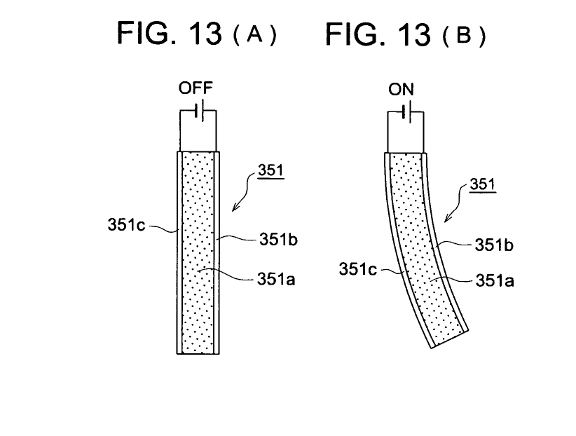

Herein, an example of the principle of operation of the conductive high polymer actuator 351 is shown in FIG. 13.

In the conductive high polymer actuator 351, gold is plated on both surfaces of the ion conductive high polymer gel 351a formed of positive ion or water molecule, and metal films 351b, 351c are formed, and the voltage of, for example, about 2V is applied on metal films 351b, 351c through the switch, not shown. Then, the conductive high polymer actuator 351 is bent on the side of the metal film 351b connected to the positive electrode side from the condition of FIG. 13(A) to the condition of FIG. 13(B).

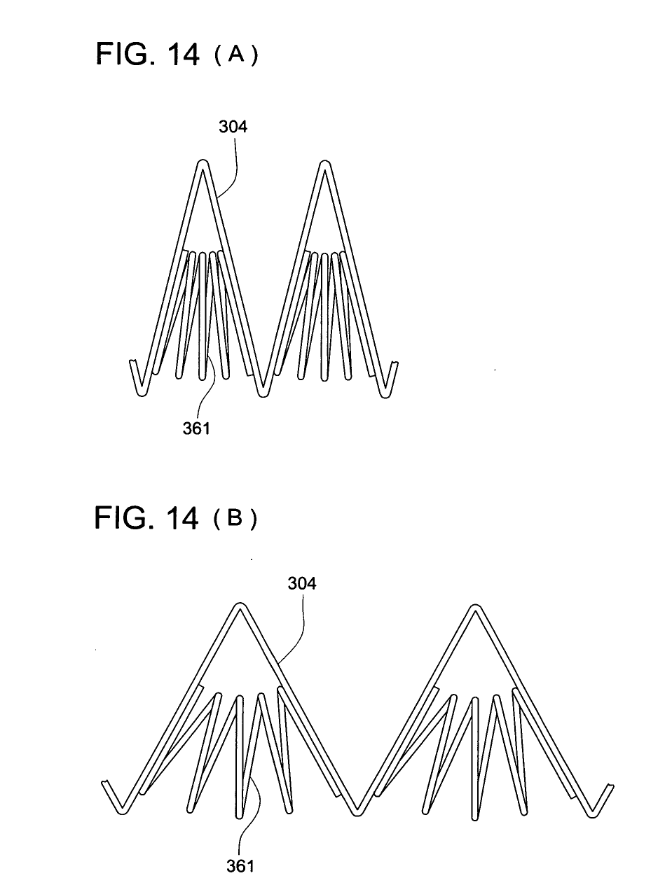

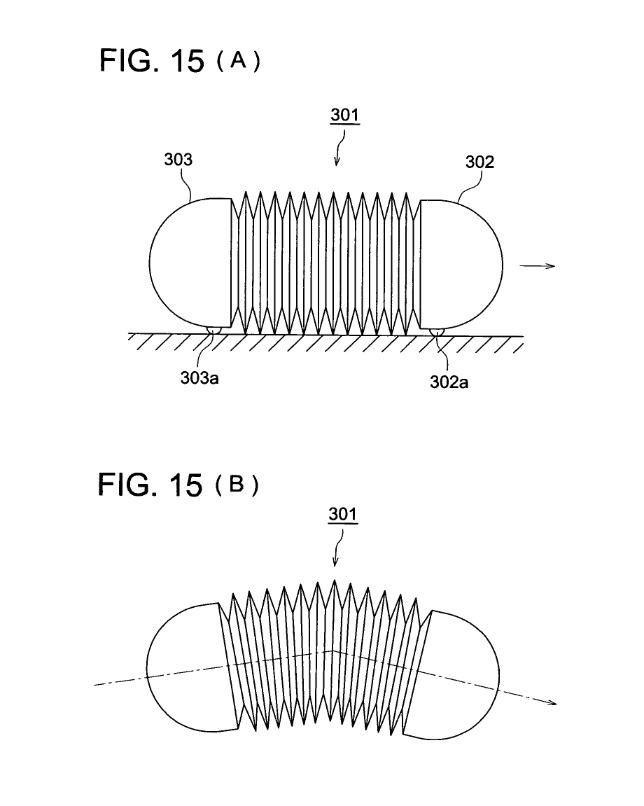

Further, as the actuator for moving the bellow group, the shape memory alloy may also be used, and this one example is shown in the enlarged view of FIG. 14.

FIG. 14(A) is a view of the condition that the bellow part is contracted, FIG. 14(B) is a view of the condition that the bellow part is stretched, and numeral 361 is the shape memory alloy. When the voltage is not applied on the shape memory alloy 361, it is contracted as shown in FIG. 14(A), and the bellow part 304 is also contracted. However, when the voltage is applied on the shape memory alloy 361, it Is heated, it is stretched as shown in FIG. 14(B), and the bellow part 304 is also stretched.

Other than that, as the actuator for moving the bellow group, the piezo-electric element, or micro-mini motor may also be used.

Further, the capsule type medical device 301 shown in FIG. 11 is an embodiment which linearly self-runs, however, the universal joints shown in FIGS. 3-5 are housed in the capsule type medical device 301, and the capsule type medical device 301 may also be structured so that it is flexible in the length direction, and is moved in the body by the fibrillation, and can pass a position which is narrow and bending at many times like as the small bowel. Further, the drive member shown in FIG. 9 is housed in the capsule type medical device and by the transmission from the outside, the capsule type medical device may also be bent in a predetermined direction and self-run.

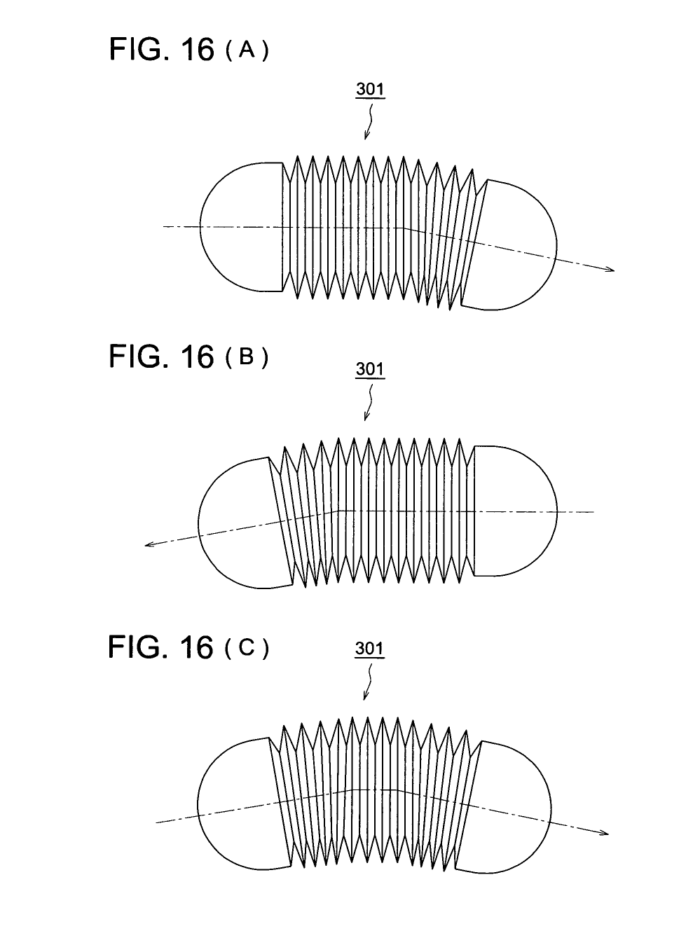

FIG. 15 is a view of the capsule type medical device 301 in which one universal joint is housed, FIG. 15(A) is its side view, and FIG. 15(B) is its upper surface view. In addition that the guide part 302a is provided on the bottom part of the front part 302, the guide part 303a is provided on the bottom part of the rear part 303, and it may also be bent as in FIG. 15(B) and self-run.

Hereupon, the guide parts 302a, 303a may also be formed like as the rotating wheel.

FIG. 16 is an upper surface view of the capsule type medical device 301 in which 2 universal joints are housed. As same as in FIG. 15(A), it has the guide parts 302a, 303a. When the capsule type medical device 301 advances, the universal joint positioned forward as shown in FIG. 16(A) is bent, and when it goes backward, the universal joint positioned backward as shown in FIG. 16(B) may be bent. Further, as shown in FIG. 16(C), both of the universal joint positioned forward and the universal joint positioned backward are bent, and may also be gone forward, and backward.

Of course, the number of the universal joint is not limited to one or two, but, 3 or more joints may also be provided.

### The Fifth Embodiment

Referring to the drawing, the fifth embodiment according to the capsule type medical device of the present invention will be described.

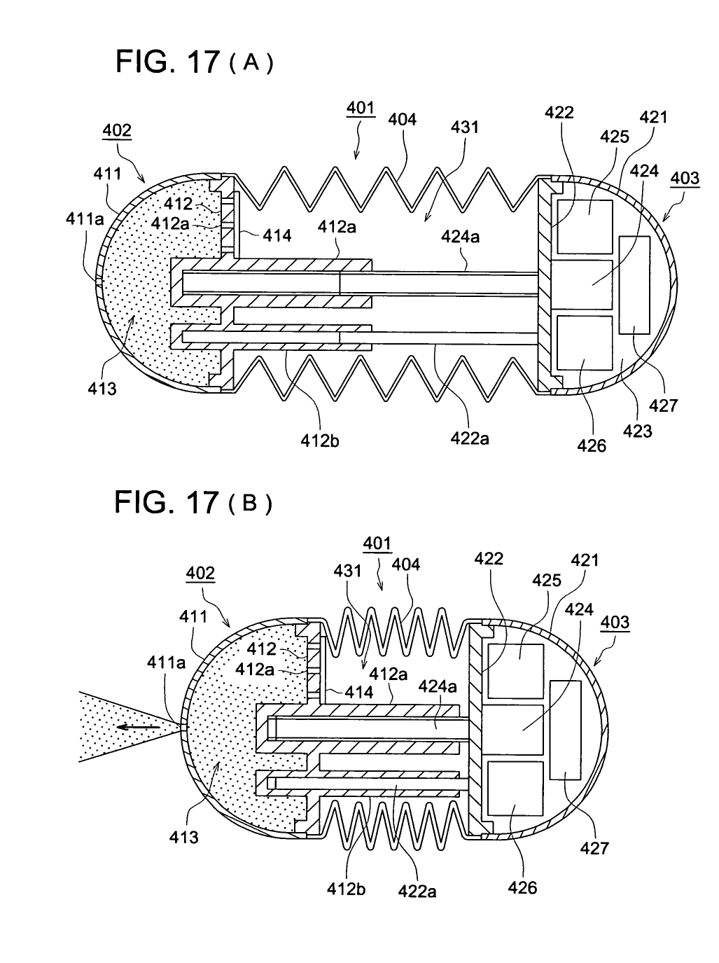

Initially, referring to FIG. 17, the capsule type medical device for injecting the medicine in the organ for cure will be described.

FIG. 17(A) is a sectional view of the capsule type medical device when it is not operated, and initially, based on this view, description will be made.

The present capsule type medical device 401 is structured by the front part 402, rear part 403, and the bellow part 404 connecting the front part 402 and the rear part 403.

In the front part 402, the front part sheath member 411, and the front part substrate 412 for holding the front part sheath member 411 are provided, and the medicine is accommodated in the about semi-spherical accommodation part 413 formed in the water-tight condition by the front part sheath member 411, and the front part substrate 412.

In the rear part 403, the rear part sheath member 421, and the rear part substrate 422 for holding the rear part sheath member 421 are provided, and the micro-mini motor 424 (drive part), transmission and reception section 425, control section 426 and battery 427 are housed in the about semi-spherical accommodation part 423 formed in the water-tight condition by the rear part sheath member 421, and the rear part substrate 422.

Hereupon, the screw shaft 424a having the male screw is protruded to the front part side from the micro-mini motor 424, and the male screw of the screw shaft 424a is engaged with the female screw of the female screw part 412a provided on the rear part side of the front part substrate 412.

The transmission and reception section 425 has the antenna and transmits the wireless signal, and makes find the position of the capsule type medical device at the outside the body, or receives the wireless signal from the outside of body so that the medicine is injected as will be described later.

The control section 426 is CPU for controlling the micro-mini motor 424 or the transmission and reception section 425.

The battery 427 is the power source of the micro-mini motor 424, the transmission and reception section 425, and the control section 426.

Further, the guide shaft 422a is protruded from the rear part substrate 422 to the front part side, and the deep hole of the deep hole part 412b protruded to the rear part side from the front part substrate 412 and the guide shaft 422a are engaged. Then, two sets of such a guide shaft 422a and deep hole part 412b are provided, both of which have the phase of about 180° in the peripheral direction of the capsule type medical device.

Further, a plurality of small holes 412c are provided in the front part substrate 412, and the small hole 412c is covered by the filter 414 having the mesh by which the powder is not passed but the air can be passed.

Next, the injection action of the medicine by the capsule type medical device 401 will be described.

Initially, when the tested subject takes down the capsule type medical device in the condition of FIG. 17(A) from the direction of the front part 402, the capsule type medical device passes the oesophagus, and arrives at the stomach, and is moved to the small bowel, and large bowel by the fibrillation. Then, from the transmission and reception section 425 of the capsule type medical device, the wireless signal is continuously transmitted to outside the body. Accordingly, by the external devices, not shown, composed of the receiving part, data processing part, display part and input part, the inspection doctor can grasp the position in the body of the capsule type medical device.

In the case where the capsule type medical device arrives at a predetermined organ, when the doctor operates the external device for injecting the medicine in this organ for cure and transmits the injection signal, the transmission and reception section 425 of the capsule type medical device receives this signal. Then, by the signal received by the transmission and reception section 425, the control section 426 controls and rotates the micro-mini motor 424.

When the micro-mini motor 424 is rotated, because the male screw of the screw shaft 424a is rotated to the female screw of the female screw part 412a, the distance of the front part substrate 412 and the rear part substrate 422 comes close. Hereupon, in this case, because the guide shaft 122a of the rear part substrate 422 is engaged with the deep hole part 412b of the front part substrate 412, there is no case where the rear part substrate 422 is rotated to the front part substrate 412 and twisted.

Hereby, as shown in FIG. 17(B), the bellow part 404 is contracted and the inside space 431 surrounded by the front part substrate 412, rear part substrate 422, and bellow part 404 is reduced, and the air in the inside space 431 is compressed. Accordingly, the inside force of the inside space 431 is increased, and the inside pressure of the accommodation part 413 is also increased through the filter 414 provided in the front part substrate 412, and the plurality of small holes 412c. Then, when the inside pressure is more than a predetermined value, the medicine accommodated in the accommodation part 413 is injected from the communication hole 411a provided in the front part sheath member 411.

Accordingly, the medicine accommodated inside can be directly injected and the cure can be made.

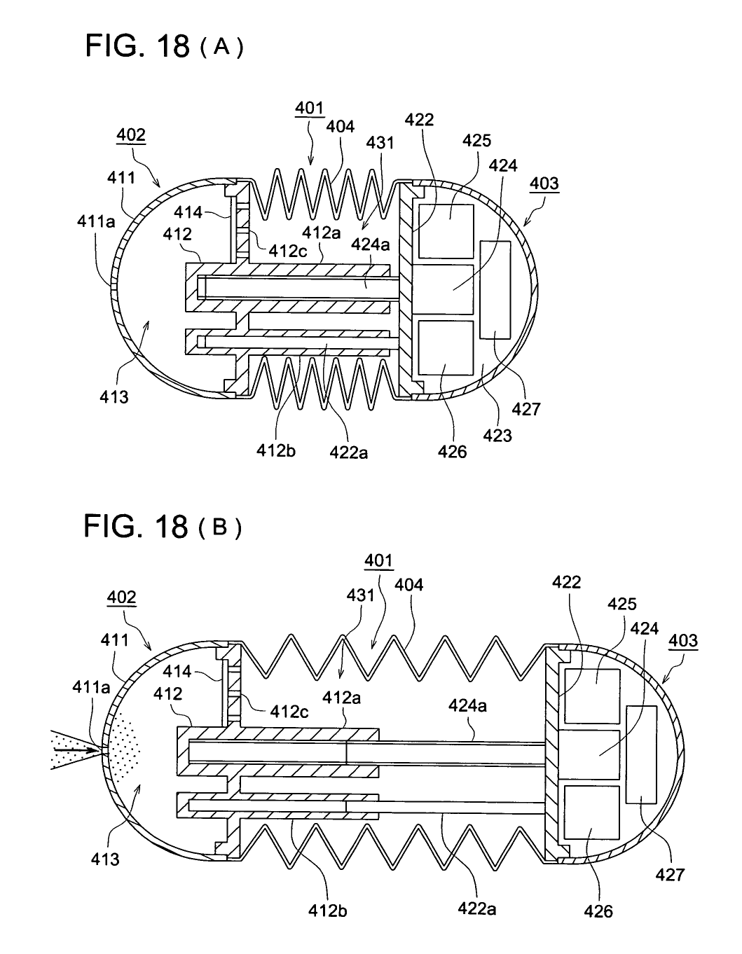

Further, inversely structured to above description, the body fluid in the organ can be sampled for the inspection. This will be described based on FIG. 18. However, because the basic structure is the same as the structure for injecting the medicine as shown in FIG. 17, the description for the same structure will be neglected.

In FIG. 18, the most different point from FIG. 17 is a point that, when the tested subject takes down the capsule type medical device 401, the bellow part 404 is contracted as in FIG. 18(A). Then, in a predetermined organ, the bellow part 404 is stretched as in FIG. 18(B). Hereby, the inside space 431 is increased, and the air in the inside space 431 is swelled. Accordingly, the inside pressure of the inside space 431 is reduced, and the inside pressure of the accommodation part 413 is also decreased through a plurality of small holes 412c provided in the front part substrate 412 and the filter 414. Then, when the inside pressure is less than a predetermined value, the body fluid in the organ enters into the accommodation part 413 from the communication hole 411a.

Accordingly, the body fluid in an arbitrary organ can be sampled and inspected.

Hereupon, as the drive part for stretching the bellow part 404, the conductive high polymer actuator, shape memory alloy, piezo-electric element may also be used other than the micro-mini motor.

Further, in the case of the structure in which the capsule type medical device injects the medicine or samples the body liquid, the image pick-up apparatus for image picking-up in the organ, may also be provided.

Further, the capsule type medical device does not have only one of the moving structure as described above, the structure for injecting the medicine, the structure for sampling the body fluid, however, it may also simultaneously have

two or three of these structures.

Other than that, in each of above described capsule type medical device 301, 401, although not shown, there is no hinder when the capsule type medical device is taken down, it is desirable that the bellow parts 304, 404 are sheathed by the sheath member formed of the material dissolved by the body fluid in the organ. As the material, for example, gelatin formed of the bone of caw, and the skin of pig or oblate formed of agar and farina is used.

Further, when the electric power is supplied to the power receiving coil provided in the capsule type medical device from the coil arranged outside the body by the electromagnetic induction, the batteries 322, 427 are unnecessary.

Further, the posture sensor is housed in the rear parts 303, 403, and the posture of the capsule type medical device may also be transmitted to the external device by the transmission and reception sections 321, 425.

Further, it may also be allowable that CPU in which the stretching and contracting motion of the bellow parts 304, 404 is written as the program is housed in the capsule type medical device, and the apparatus is automatically moved in the body, the medicine is injected, or the body fluid is sampled.

What is claimed is:

1. A capsule type medical device to conduct an operation in an internal organic portion of a body, comprising:
a capsule body to move in the internal organic portion; and
an operating member incorporated in the capsule body and to conduct the operation in the internal organic portion;
wherein the capsule body includes a bellow section formed on a peripheral surface of the capsule body such that the bellow section stretches or contracts in a longitudinal direction of the capsule body.

2. The capsule type medical device of claim 1, wherein the bellow section is in a stretching condition on a free condition thereof and the bellow section is wrapped by a covering member soluble in the internal organic portion on a contracted condition on which the bellow section is contracted from the free condition.

3. The capsule type medical device of claim 1, wherein the operating member comprises an image pick-up system to pick up an image and an image processing section to conduct an image processing for the picked-up image and the bellow section comprises a first bellow to surround at least the image pick-up system and a second bellow to surround at least the image processing section.

4. The capsule type medical device of claim 3, wherein the operating member comprises a first holding member to hold the image pick-up system and a second holding member to hold the image processing section and the first holding member is connected with the second holding member by a universal joint.

5. The capsule type medical device of claim 1, wherein the operating member comprises an image pick-up system including a first lens located on a front side of the capsule body and a second lens located in the capsule body and a driving member to drive the bellow section to stretch or contract to move the front side of the capsule body so that the first lens is shifted in an optical axis direction so as to change a distance between the first lens and the second lens.

6. The capsule type medical device of claim 5, wherein the driving member changes an object distance of an object to be focused by changing the distance between the first lens and the second lens.

7. The capsule type medical device of claim 5, wherein the driving member changes a focal length of the image pick-up optical system by changing the distance between the first lens and the second lens.

8. The capsule type medical device of claim 1, wherein the operating member comprises an image pick-up system to pick up an image and an image processing section to conduct an image processing for the picked-up image, the capsule body comprises a front body to accommodate the image pick-up optical system and a rear body to accommodate an image processing section, the bellow section is located between the front and rear bodies to joint the front and rear bodies and the image pick-up optical system and the image processing section are jointed swingingly by a joint member, and wherein the operating member further comprises a driving member to drive the bellow section in such a way that a part of the bellow section stretches and another part of the bellow section contracts so that the front body is relatively inclined to the rear body.

9. The capsule type medical device of claim 8, wherein the joint member is a universal joint.

10. The capsule type medical device of claim 8, wherein the driving member includes an ultra miniature motor.

11. The capsule type medical device of claim 1, wherein the bellow section comprises a plurality of sub bellows and each of the plurality of sub bellows is adapted to stretch or contract independently from others so that the capsule body is moved by itself.

12. The capsule type medical device of claim 11, wherein the operating member comprises an actuator to stretch or contract each of the plurality of sub bellows independently in such a way that only a first sub bellow positioned at the leading edge in an advancing direction is stretched on a condition that all of the among the plurality of sub bellows are contracted so that the leading edge of the capsule body is moved forward, next, the first sub bellow is contracted and the second sub bellow positioned behind the first sub bellow is stretched, in succession, the driving member repeats the contraction and the stretch in such a way that the proceeding sub bellow is contracted and the succeeding sub bellow positioned behind the proceeding sub bellow is stretched, and after a last sub bellow positioned at the trailing edge in the advancing direction is stretched, when the last sub bellow is contracted, the trailing edge part of the capsule body advances forward.

13. The capsule type medical device of claim 12, wherein the total length of the plurality of sub bellows in a stretched condition is shorter than the total length of the plurality of sub bellows in a contracted condition at a time of a standstill.

14. The capsule type medical device of claim 11, wherein the operating member is separated into at least two units and the two units are jointed swingingly with a joint member.

15. The capsule type medical device of claim 14, wherein the operating member comprises a driving section to drive the plurality of sub bellows in such a way that a part of the plurality of sub bellows stretches and another part of the plurality of sub bellows contracts so that one of the two units is relatively bent to the other one of the two units around the joint member.

16. The capsule type medical device of claim 11, wherein the operating member comprises at least an image pick-up lens, an illumination section to illuminate an object, an image pick-up element to photo-electrically convert a picked-up image, an image processing section to conduct an image processing to the photo-electrically converted image, and a transmission and reception section to transmit the image processed image to an external device.

17. The capsule type medical device of claim 12, wherein the actuator is a conductive high polymer actuator.

18. The capsule type medical device of claim 12, wherein the actuator comprises a shape memory alloy to drive the plurality of sub bellows.

19. The capsule type medical device of claim 1, wherein the operating member comprises a storage section and a driving member to drive the bellow section to stretch or contract and the capsule body includes a through hole communicating with the storage section.

20. The capsule type medical device of claim 19, wherein when the driving member drives the bellow section to contract, a medicine stored in the storage section is jetted from the storage section through the hole to the internal organic portion.

21. The capsule type medical device of claim 19, wherein when the driving member drives the bellow section to stretch, a body liquid is sampled from the internal organic portion through the hole to the storage section.

22. The capsule type medical device of claim 19, wherein the operating member comprises at least an image pick-up lens, an illumination section to illuminate an object, an image pick-up element to photo-electrically convert a picked-up image, an image processing section to conduct an image processing to the photo-electrically converted image, and a transmission and reception section to transmit the image processed image to an external device.

23. The capsule type medical device of claim 19, wherein the driving member comprises an ultra miniature motor.
